(19) United States
(12) Patent Application Publication
(10) Pub. No.: US 2024/0102990 A1
(43) Pub. Date: Mar. 28, 2024

(54) DEVICE FOR ENHANCED DETECTION OF CELLULAR RESPONSE

(71) Applicant: LUMACYTE, INC., Charlottesville, VA (US)

(72) Inventors: Sean Hart, Charlottesville, VA (US); Colin Hebert, Charlottesville, VA (US)

(21) Appl. No.: 18/275,997

(22) PCT Filed: Feb. 8, 2022
(86) PCT No.: PCT/US2022/015576

§ 371 Date: Aug. 4, 2023

Related U.S. Application Data
(60) Provisional application No. 63/146,969, filed on Feb. 8, 2021.

(51) Int. Cl. G01N 33/50; C12M 1/12; C12M 1/26; C12M 1/34; C12M 3/06

(52) U.S. Cl. CPC G01N 33/5008; C12M 23/16; C12M 25/04; C12M 33/04; C12M 41/46; G01N 33/5091

(57) ABSTRACT

Improved biomanufacturing devices, apparatuses and methods for using the same for monitoring biologic products such as biologies, vaccines, cell and gene therapies for viral safety and identification of cytopathic effect, In certain embodiments, the invention described herein enables the objective analysis of adventitious agents including adventitious viruses, bacteria and mycoplasma.

### FIELD OF THE INVENTION

Embodiments of the present disclosure relate generally to a device designed to expose cells to a proportionally larger volume of sample fluid containing trace amounts of analytes to be detected by measuring cellular response or other properties. The embodiments described herein include the enablement of enhanced performance and objective analysis of various adventitious agents, including adventitious viruses, bacteria, and mycoplasma. The detection of any of these species is collectively referred to adventitious agent testing (AAT).

### SUMMARY OF THE INVENTION

Currently available procedural and analytical methodologies for the characterization of biological cells and systems such as infectivity assays (e.g., neutralization assays, TCID50 and clinical sample manipulation) require extensive dilutions, potentially detrimental tagging procedures and yield highly variable results making inter- and intra-experimental and trial comparisons challenging and downstream cellular applications limited. Specifically, the testing of biologic products (biologics, vaccines, cell and gene therapies) for viral safety is particularly difficult and relies on visual identification of cytopathic effect in a complement of cell lines monitored over 14 days with an additional 14 days to catch any additional signs of infectious viral agents. Such viral safety testing, also referred to as adventitious agent testing (AAT) is a critical part of the release process for biologic drugs, treatments, and therapies. The ability to decrease the assay time would expedite product release and an increase in sensitivity would result in a better assay making these life saving products safer and ensuring their continuous availability. The invention disclosed herein seeks to accomplish all of these goals through the use of fluidic devices designed to expose a specific number of reporter cells (from relevant cell lines) to a volume of bioreactor fluid (condition media). The exposed cells are incubated with the condition media and then released for later analysis using laser force cytology or other detection methodologies.

The current invention overcomes limitations of the prior art by providing a novel device to enhance the detection of adventitious agents.

### BRIEF DESCRIPTION OF THE DRAWINGS

FIG. 1 Provides one embodiment of the overall device, showing a region of cell confinement, one or more fluid reservoirs, and a network of channels connecting them.

FIG. 2 Provides an alternate embodiment that incorporates a loop for pumping sample fluid such as cell culture medium through the cell confinement region.

FIG. 3 Provides further details of the operations of the device, including multiple reservoirs to introduce reagents in addition to cell culture medium. These reagents could be used to release cells for direct sampling by a single-cell analysis or other instrument.

FIG. 4 An embodiment where a manifold is used to deliver fluid to the cell containing regions

FIG. 5 An embodiment where multiple cell containing regions are used either in series or in parallel.

FIG. 6 Provides one embodiment of an aspect of the device for introducing cultured cells into the device using a disposable insert containing pre-cultured cells, where the fluid layer is near the surface where the insert attaches.

FIG. 7 Provides one embodiment of an aspect of the device for introducing cultured cells into the device using a disposable insert containing pre-cultured cells, where the fluid layer enters through the upper region of the insert and fluid travels down through and out of the cell containing region.

FIG. 8. Provides one embodiment of an aspect of the device for introducing cultured cells into the device using a disposable insert containing pre-cultured cells, that is screwed or press-fit into the plastic fluidic device. The fluid layer enters through the upper region of the insert and fluid travels down through and out of the cell containing region.

FIG. 9. Depicts an oil containing syringe into which condition media is drawn and is encapsulated by the oil. This can remain separated from the plastic or glass walls of the syringe potentially avoiding viral losses due to adherence to the vessel walls.

FIG. 10. Provides an embodiment of the device where the cell confinement region is narrower that the surrounding channels in order to focus the analyte onto the cells. A permeable membrane or barrier confines the cell supports but allows fluid and analyte to flow freely. Several embodiments of the cell confinement region are also shown.

FIGS. 10A and 10B. Provide various embodiments for removing cell supports from the confinement region

FIG. 11 provides an embodiment that employs forces (optical, magnetic, electrokinetic, etc.) to contain the cells in the central region while fluid flow passes over them.

FIG. 12 provides an embodiment of the device designed to use differential forces to concentrate analyte to the cells located in the confinement region and repel them from the channel and other walls.

FIG. 13. Hydrodynamic flow focusing of an aqueous phase within an oil phase to concentrate condition media or other fluid to be tested withing the fluidic device and away from the device walls.

### DETAILED DESCRIPTION OF THE INVENTION

The present invention is described with reference to particular embodiments having various features. It will be apparent to those skilled in the art that various modifications and variations can be made in the practice of the present invention without departing from the scope or spirit of the invention. One skilled in the art will recognize that these features may be used singularly or in any combination based on the requirements and specifications of a given application or design. One skilled in the art will recognize that the systems and devices of embodiments of the invention can be used with any of the methods of the invention and that any methods of the invention can be performed using any of the systems and devices of the invention. Embodiments comprising various features may also consist of or consist essentially of those various features. Other embodiments of the invention will be apparent to those skilled in the art from consideration of the specification and practice of the invention. The description of the invention provided is merely exemplary in nature and, thus, variations that do not depart from the essence of the invention are intended to be within the scope of the invention.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Unless otherwise defined, all technical and scientific terms used herein have the same meaning as would be commonly understood or used by one of ordinary skill in the art encompassed by this technology and methodologies.

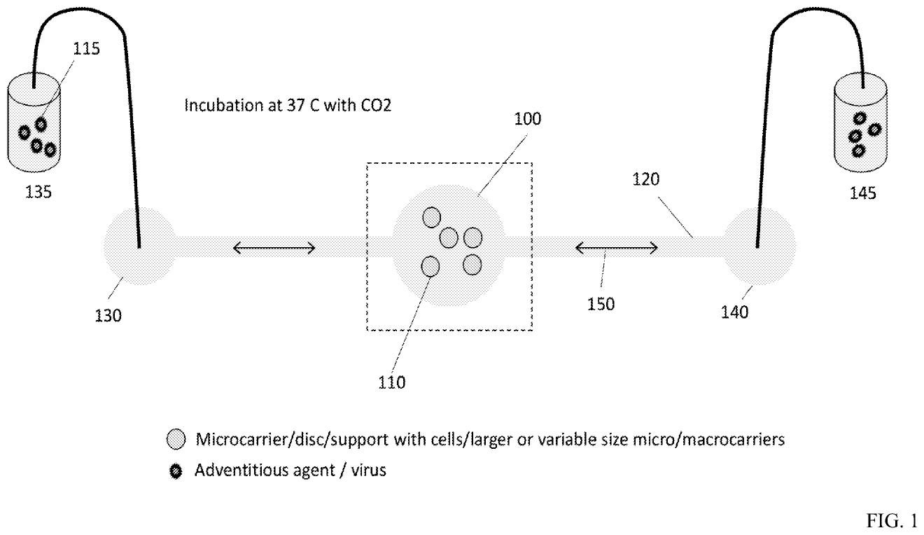

FIG. 1 shows reservoirs (135, 145) for conditioned media or other fluids to be tested for adventitious agents or other sample analytes (115), including viruses, bacteria, mycoplasma, or toxins, and a fluidic device with a cell confinement region (100) for exposure of cells attached to the surface of the device, present on cell supports (110), such as microcarriers, cell growth discs, or other cell growth carriers. These supports could be of various sizes, compositions, and structures. These supports could be dissolvable via chemical or physical means, and amenable to movement using forces such as optical, acoustic, or magnetic. Fluid flow (150) would be used to pass the conditioned media or other fluids over the cell confinement region in order to elicit a cellular response.

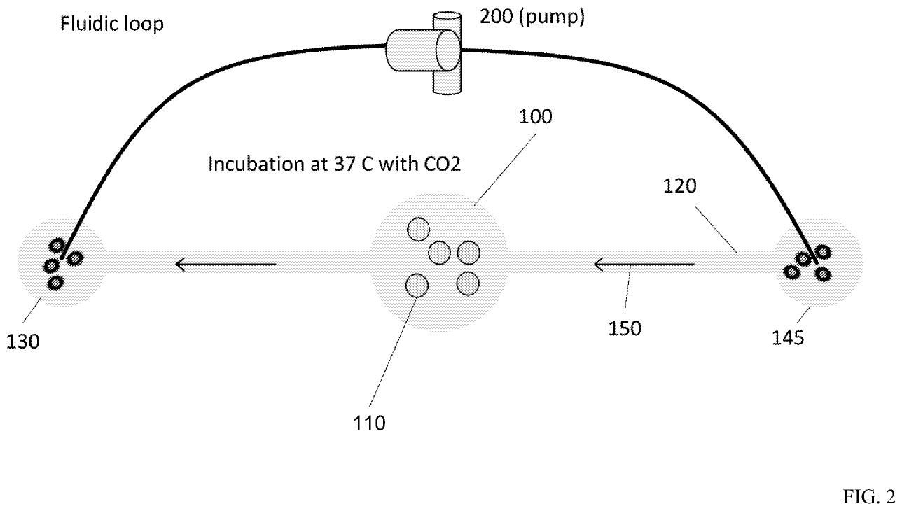

FIG. 2 shows reservoirs (135, 145) for condition media or other fluids to be tested for adventitious viruses, and a fluidic device with a region (100) for exposure of cells attached to the surface of the device, present on microcarriers (110), or cell growth discs, or other cell growth carriers. The addition of a pump (peristaltic, or other type) allows circulation of the media over the cellular region.

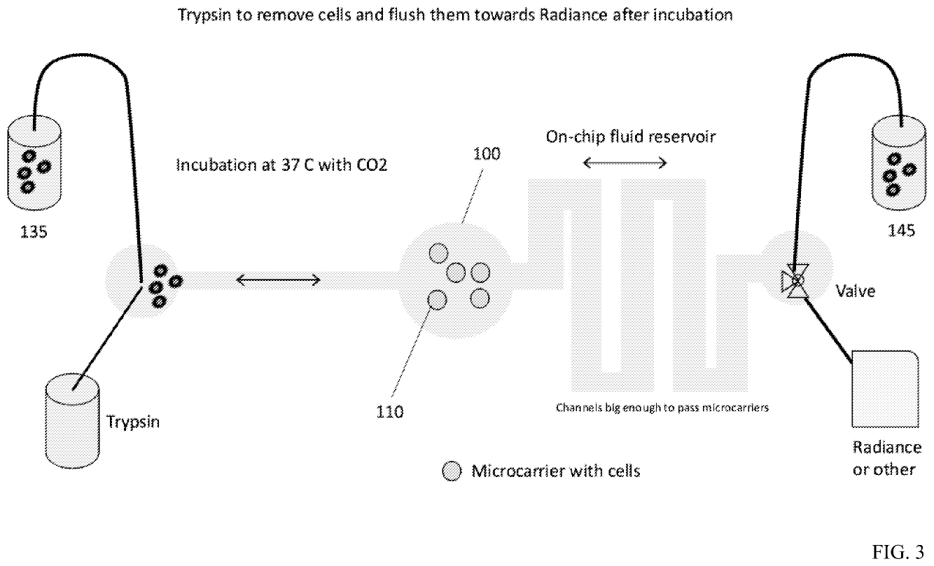

FIG. 3 shows reservoirs (135, 145) for condition media or other fluids to be tested for adventitious viruses, and a fluidic device with a region (100) for exposure of cells attached to the surface of the device, present on microcarriers (110), or cell growth discs, or other cell growth carriers. The addition of other reservoirs to allow reagent addition such as trypsin for removal of cells from growth carriers. Additionally, the presence of a serpentine large enough to accept microcarriers and serve as a detachment region prior to a value to remove cells from the device to deliver to an analytical instrument including but not limited to single cell analysis (optical forces, cytometry), nucleic acid analysis (PCR or sequencing), or protein analysis (mass spectrometry).

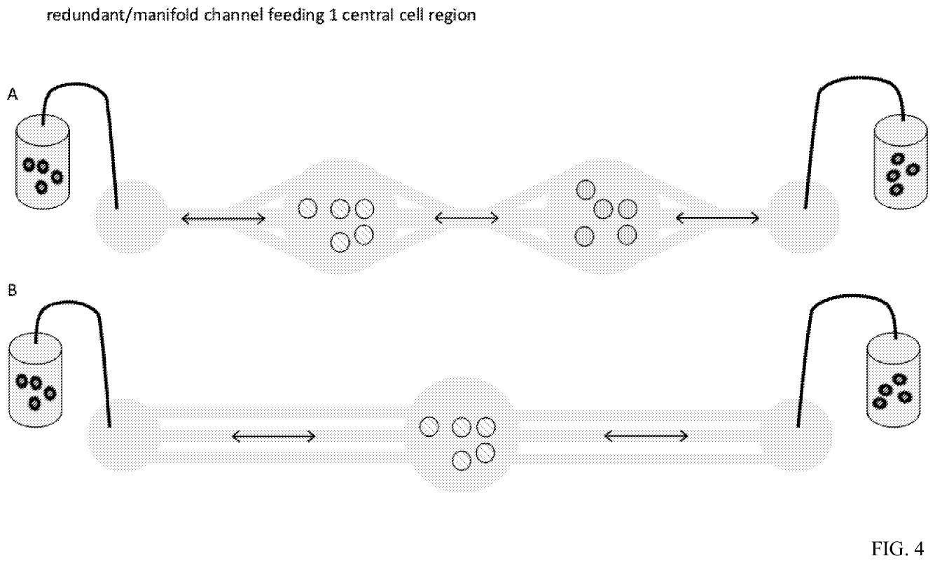

FIG. 4. Similar in concept to earlier figures but includes manifolds to deliver more fluid to the cellular regions at a lower velocity, while utilizing smaller channels that will not allow escape of the microcarriers and additionally limits clogging. A) depicts a manifold from the main channel to the cell exposure region. B) shows channels connecting the input reservoirs directly to the cell exposure region. While 3 channels are shown, any number could be used in practice from micro- to milli-fluidic sizes.

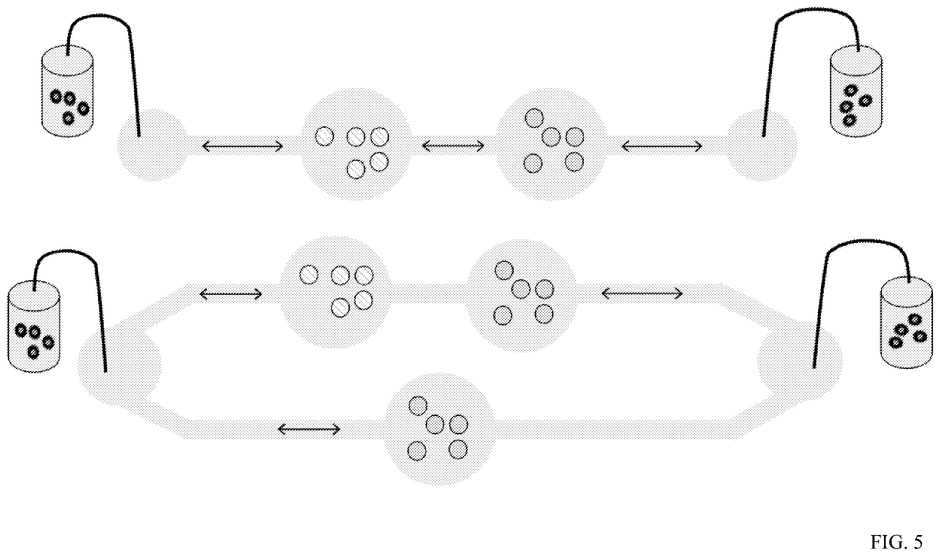

FIG. 5 shows the use of multiple different cell types in one device either in series or in parallel.

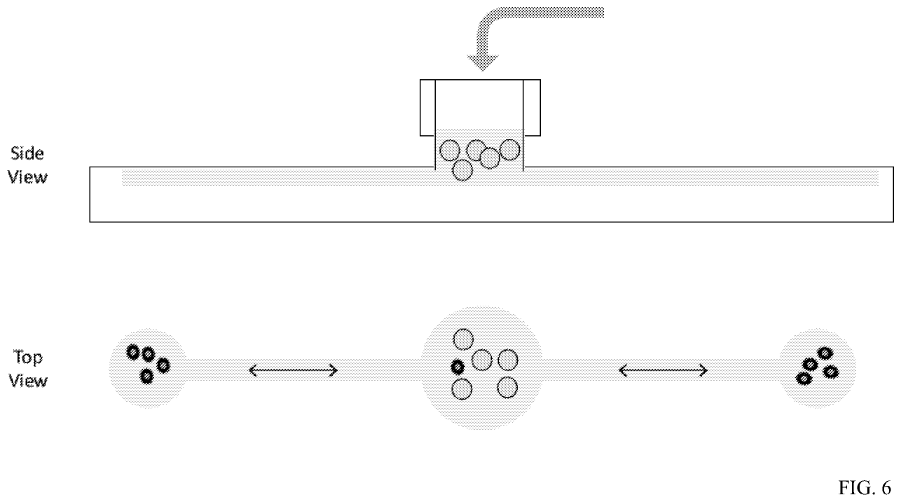

FIG. 6. Depicts a fluidic chip with a region for cells and fluid to be inserted from above and the cell carriers settle down into the fluid flow. The cells and fluid to be added could be frozen and would thaw and be used directly in the fluidic device before being exposed to fluid or condition media.

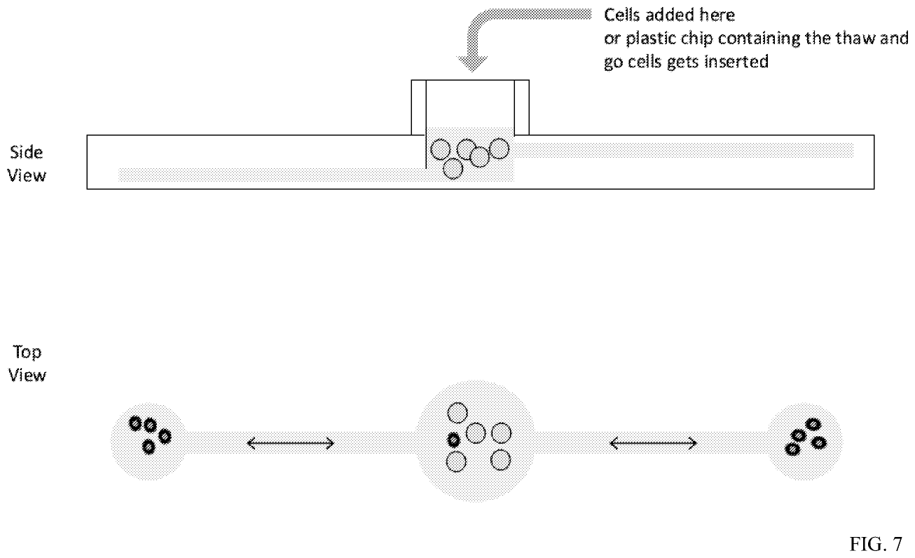

FIG. 7. Similar to FIG. 6 except that the fluid flow enter the top region of the cell holder (700) and flow downward across the cell carriers and out of the holder (700) region.

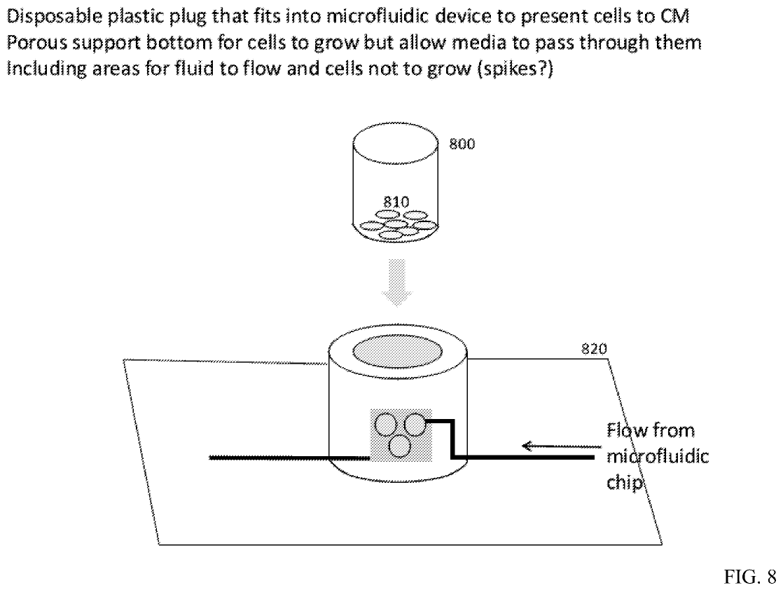

FIG. 8. Concept of operations for the cell holder (800) and an insert containing the cells such that the insert would be disposable and the fluidic chip (820) could be reusable or disposable. The cylindrical cell holder would screw into the fluidic device and make fluidic contact between the fluid in the device and the cells (810) in the holder (800). Flow would again enter the holder and move across the cells downwards to the exit on the other side ensuring good residence time with the cells (810)

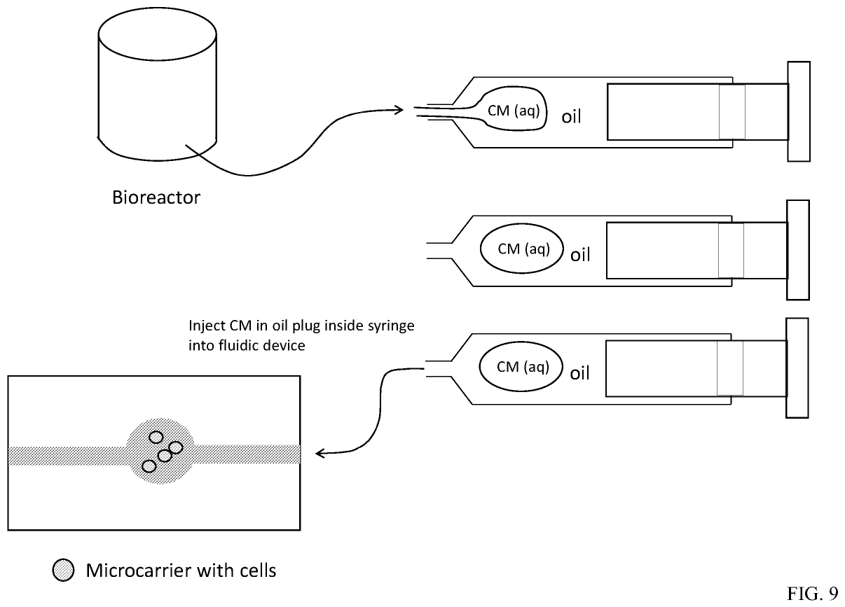

FIG. 9. Syringe to withdraw CM fluid into oil phase inside syringe or in a fluidic device. This keeps CM and virions from contacting wall where they can adhere removing them from the solution. This could also be achieved using a hydrophobic coating as well.

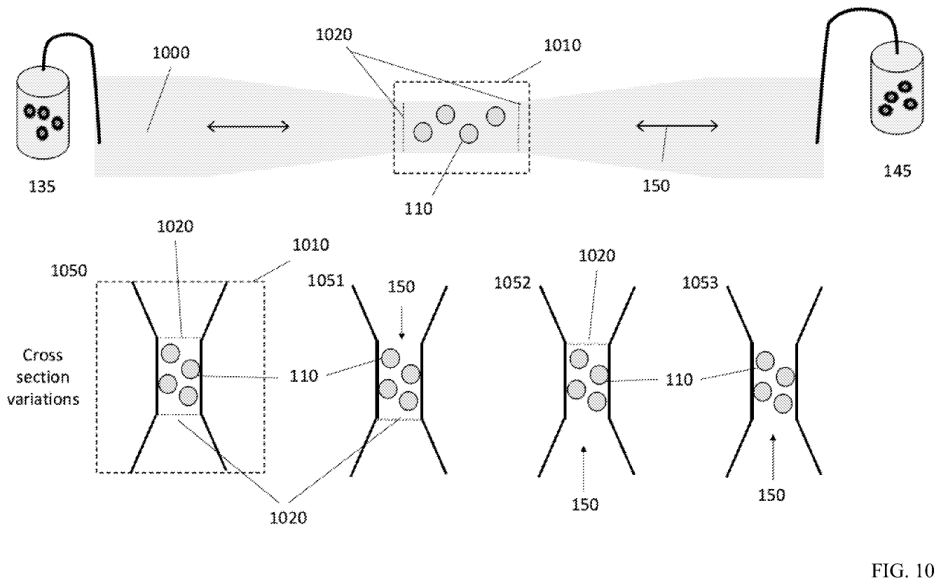

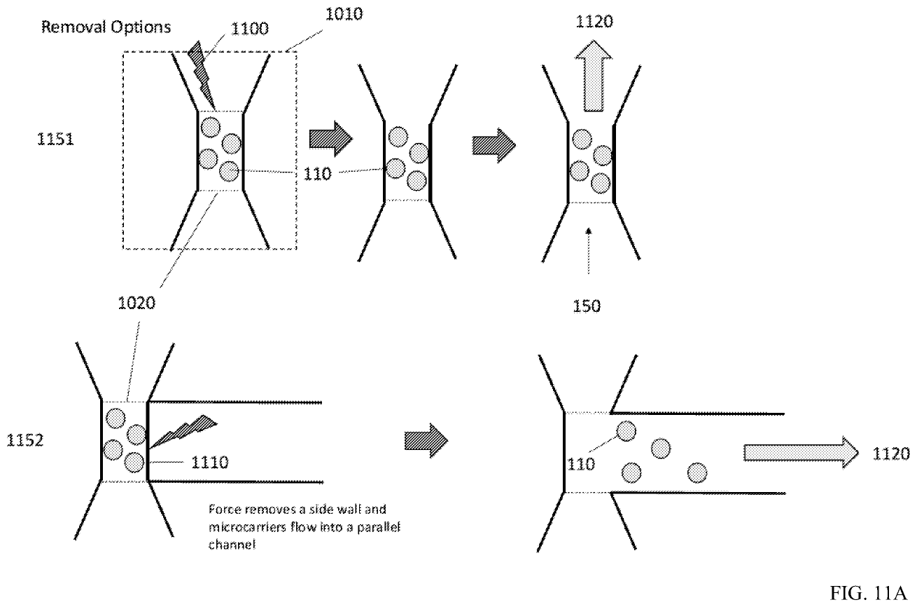

FIG. 10 shows an alternate embodiment in which the channels (1000) have a larger diameter than the cell supports. This could help concentrate any sample analyte into the cell confinement region (1010) and to the cell supports (110). An additional feature of this embodiment is the use of one or more semi-permeable barriers (1020) in order to confine the cell supports to the to the cell confinement region. These barriers could be a membrane, frit, filter, or geometric feature of the channel such as a pillar or weir, so long as they prevent the cell supports from leaving the confinement region but allow liquid including sample analyte to flow freely through the cell confinement region and interact with the cells on the cell supports. Alternatively, the cell supports could be designed in such as way as to attach or both to the channel wall or floor and thus be immobilized without the use of the barrier (1020). A chemical, physical, or other bond could be used to affix the cell supports and prevent them from leaving the cell confinement region. Other features and embodiments listed earlier figures could also be used with this design, including but not limited to the looped pumping in FIG. 2, the parallel channels and multiple cell types shown in FIGS. 4 and 5 and the cell insertion devices shown in FIGS. 6-8. Several embodiments of the cross section of the cell confinement region are also shown in the bottom portion of the figure. Variation 1050 has the barrier (1020) on both sides of the cell confinement region, and thus can be oriented either horizontally or vertically with respect to gravity and flow can be in either direction. Variation 1051 has the barrier (1020) on only 1 of the sides and thus the particles must be actively held against the barrier either by fluid flow (150), gravity (which is accomplished through orienting the channel vertically with respect to gravity and with the barrier on the bottom as shown), or some other force such as a optical, magnetic, acoustic, or fluidic. Variation 1052 also has only one barrier (1020) with the cell confinement region oriented vertically with respect to gravity, but the barrier is on the top, so the fluid flow (150) must overcome the gravitational settling force in order to keep the cell supports (110) confined to the confinement region (1010). The design facilitates easy removal of the cell supports for cell recovery by stopping the fluid flow and allowing the cell supports (110) to settle against the force of gravity. Finally, variation 1053 has no barriers and is oriented vertically with respect to gravity. In this case, the settling force of the cell supports (110) is exactly balanced by the force of the fluid flow and their net movement is zero or close to zero. This keeps them within the confinement region (1010).

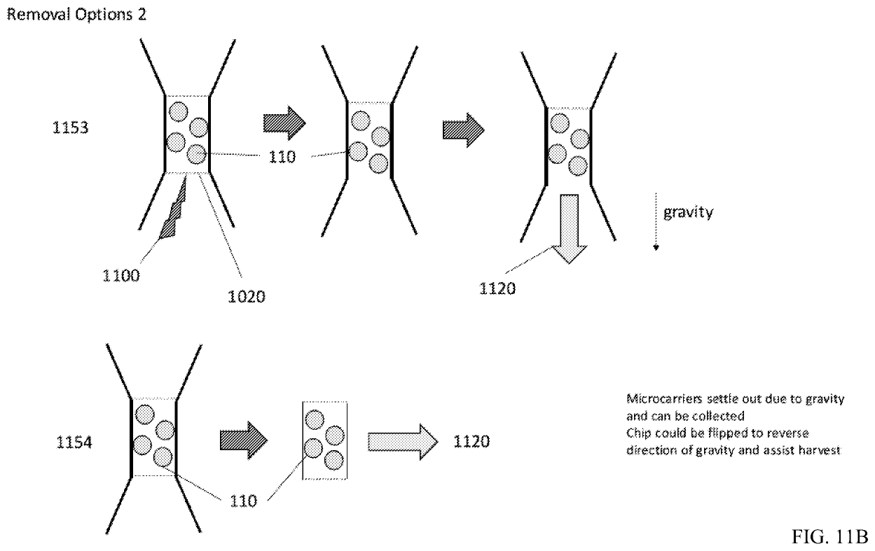

FIGS. 11A. and 11B show various embodiments for harvesting the cell supports (110) from the confinement region (1010). In embodiment 1151, a force 1100 destroys, removes, or enables the removal of one of the barriers 1010. This force could be optical, electrical, mechanical, thermal, acoustic, or magnetic. Once one of the barriers has been removed, fluid flow 150 can then push the cell supports (110) out of the confinement region and to the harvest region or device (1120). In embodiment 1152, the force (1100) removes one of the channel walls (1110) orthogonal to the fluid flow. This creates a new side channel that can be used to transport the cell supports to harvest (1120). In FIG. 11B, embodiment 1153 shows the force (1100) removing the bottom barrier (1020). This allows the cell supports (110) to settle due to gravity and be easily transported to the harvest region (1120). Finally, in embodiment 1154, the entire cell confinement region is moved out of the device and transported to the harvest region or device (1120).

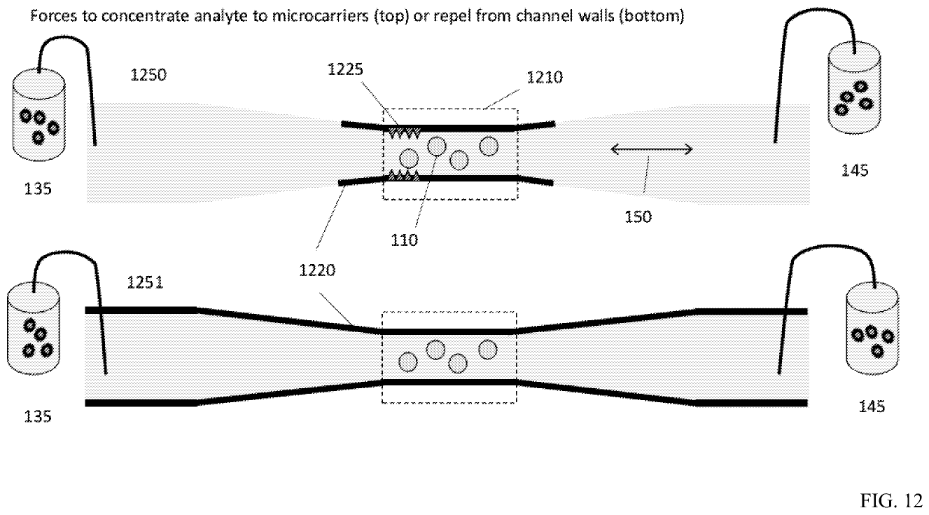

FIG. 12 demonstrates the concept of using one or more external forces to enhance the performance of the device. This force could be electrical, magnetic, optical, acoustic, chemical, thermal, or fluidic. Embodiment 1250 shows the use of a force to concentrate or preferentially locate the sample analyte (115) within the cell confinement region (1210) to increase the interaction time with the cell supports (110) to maximize the likelihood of cells responding to the analyte. This could also include physical modifications (1225) to the channel around the cell confinement region. One embodiment shown is the use of sawtooth structures to enhance dielectric focusing. In another embodiment 1251, the channel is modified in such a way as to repel the sample analyte (115), thereby reducing the probability that trace amounts of for example virus or bacteria stick to the channel walls and thus do not interact with the cells within the confinement region (1210). This could be done through the use of an active force as well as a physical or chemical coating (for example hydrophobic) that might repel the sample analyte. FIG. 12. Embodiment has an interaction region/zone that would use external force (optical, magnetic, electrokinetic, acoustic, or other) to position cells such that they would not be lost due to the flow of condition media or other fluid across them, backwards and forwards utilizing the pumps (135, 145).

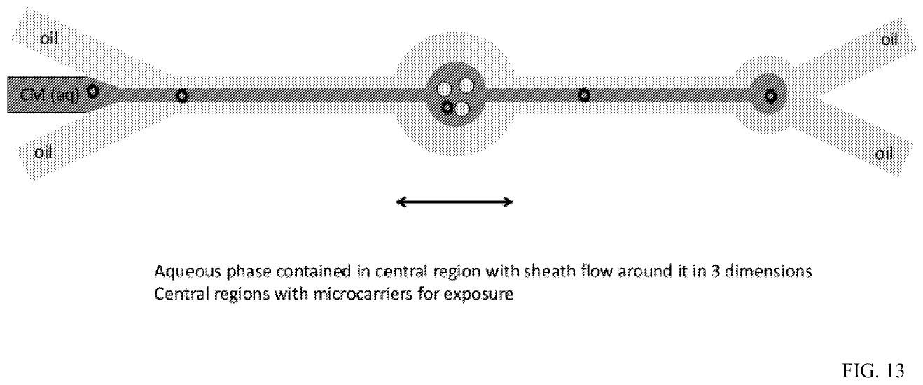

FIG. 13. The embodiment involves using 3D hydrodynamic focusing to contain an aqueous phase (condition media/test fluid) within a sheath of oil. The focused region would encounter a widening (circular, square, pyramidal, conical, or other shape) in the fluidic channel. The cells would be contained in this region by attachment to the surfaces, held in a scaffold or large microcarrier. The oil with spread to the outer regions and the aqueous phase with cover the cells and permit the exposure by flowing them backwards and forward over the cell containing region.

As is known to those skilled in the art, one serious concern associated with the manufacture of biological products such as vaccines and cell and gene therapy products, is the inadvertent introduction of adventitious agents (endogenous or exogenous). The use of optical force-based measurements, such as those obtained using LFC to detect adventitious agents (AA) in bioreactor condition media or other fluids used in biomanufacturing, is an important capability of the novel methodologies described herein. The methods of the present invention enable the critical assessment of quality and prevention of bacteria, viruses, or other replicating/living contaminants from jeopardizing the production of drug substances. The ultimate goal of advanced AAT using LFC is to thwart and limit any possible inclusion in a drug product that could lead to potential infection of patients. The overall process for using LFC for measuring viral infectivity in biomanufacturing is shown in FIG. 4 where condition media (CM) from a bioreactor or other manufacturing process is mixed with cells growing in suspension or adherent culture and incubated for a shorter period than current methods which currently take 14 days or more under FDA guidelines. The same cells are monitored using blank samples as controls. The amount of time the cells are exposed to the conditioned media can be adjusted as part of the assay optimization.

In an embodiment, the first line of defense when using LFC to monitor for AA is using CHO or another cell line used for bioproduction directly as a responsive cell that can be measured using LFC. While not all viruses cause cytopathic effects in CHO cells (and other production cell lines), many do, and this forms the basis for real-time monitoring of changes in CHO cells during production. Deviations in variables measured using LFC can be used as indicators of potential contamination by AA. This is shown in FIG. 5 where the overall strategy for AAT using Radiance™/LFC is given. CHO cells used in production are constantly monitored by a sampling system that removes cells and introduces them to Radiance™ for LFC analysis to gauge changes in their intrinsic properties as a way to monitor for AA. CPE may be visible if AA are present and this differs from any changes in LFC measured variables used to monitor protein production. Samples could also be removed from the bioreactor and run separately in Radiance™ using LFC as opposed to on-line analysis. Condition media (CM) can be removed and incubated with cells with or without concentration (e.g., centrifugation to concentrate potential AA). After an incubation period or throughout the incubation period, cells can be monitored for signs of AA. Radiance™/LFC can sort out potentially infected cells and collect them for analysis using other methods including spectroscopic (fluorescence, Raman, or other), polymerase chain reaction (PCR), next generation sequencing (NGS), mass spectrometry (MS), cytometry (flow, fluorescence, mass, or image) or other methods.

For those viruses that do not cause cytopathic effects in CHO cells, other cell lines can be used for detection. FIG. 6 shows a partial list of viruses and classifies them according to cytopathic effect and replication. This indicates that four cell lines can provide decent coverage of potential viruses: Vero cells, baby hamster kidney cells (BHK), MRC-5 cells, and Human kidney fibroblast (324K) cells. The panel is not limited to these four cell lines and other existing cell lines can be used, as well as newly developed cell lines modified for specific susceptibility.

In an additional embodiment, the methods described herein may be used to classify viruses or other AA based on a specific pattern of data. Several methods could be used for this, including artificial neural networks (ANN), pattern recognition, or other methods of predictive analytics. A specific data example of this using LFC data is shown in FIG. 22. Here, an ANN is used to classify test samples as one of three potential viruses using approximately 17 LFC parameters as the input.

In certain embodiments, to speed analysis, multiple cell lines can be run simultaneously as in vitro sentinel cell lines with condition media (CM) or another analyte. In certain embodiments, sentinel cells are cells that are susceptible to the condition (viral, bacterial, mycoplasma, infection, or other AA) being monitored or detected and their response can be measured using LFC. FIG. 7 shows a multiplexed assay using multiple in vitro sentinel cell lines in each well or biosampling system. The ability to differentiate the cells in Radiance™/LFC by parameter space or using other tags, fluorescence, visual brighfield microscopic identification, or others means would greatly increase throughput by allowing the cells to be incubated together and run at the same time. Cells engineered to have different parameters in Radiance™/LFC so they will not be confused with one another can be used to multiplex the assays. Methods to multiplex by modifying the cells to have different properties include but are not limited to: Fluorescence based—green fluorescent protein (GFP), red fluorescent protein (RFP), yellow fluorescent protein (YFP) and other genetic modifications incorporated into macrophage line or other cell lines so one can determine which one is reporting presence of cytopathic or other effect due to AA. Cells analyzed using LFC can also be labelled with, by way of example only, stain, dye, antibody conjugated bead labels, affinity bound beads or molecules, nano-particles (Au, Ag, Pt, glass, diamond, polymer, or other materials). Nanoparticles could have different shapes (spherical, tetrahedral, icosahedral, rod or cube shaped, and others) and size to accomplish two objectives: 1) varied entry into cells, and 2) changing the optical force measurable using LFC.

In certain embodiments, nanoparticles may be incubated with the cells and uptake would happen as normal for the cell type or alternatively nanoparticle uptake could be augmented chemically or physically (such as by electroporation or facilitated by liposomes) to enhance nanoparticle uptake percentages. Cells would be incubated with nanoparticles and a virus to be tested and an increased differential of viral uptake into cells would lead to a larger differential in optical forces measured using LFC, thus improving viral detection sensitivity. In alternate embodiments, nanoparticles may be incubated with the virus prior to exposure to the cells.

In additional alternate embodiments, macrophages that engulf a specified number of beads would have different properties in LFC but would still report the presence of AA. Additionally, only specific portions of the cell could be analyzed, such as the nucleus, mitochondria, or other organelles. This could be used to enhance the performance not only AA but also other cell-based assays including infectivity.

In aspects, cells may be genetically engineered to have different viral, bacterial, fungal, or other AA susceptibility for use as in vitro sentinel cells, in an embodiment, in the panel used with Radiance™/LFC would allow a tailored approach to AA detection. Incorporating or eliminating certain genes into or from the cell line may make the cell line more permissive to infection with a particular class of viruses, bacteria, or other AA, thus affording rapid detection with selectivity of pathogen type. This combined with the broad viral identification possible using LFC will allow better identification of viral, bacterial, or other type of AA.

The novel methods described herein demonstrate that AAT could occur directly on cells removed from the production bioreactor (800) through analysis immediately using LFC/Radiance™ (810) as shown in FIG. 8. For AA that do not produce CPE or other effects in the production cell line (CHO or others), additional suspension cell lines can be used in mini analytical bioreactors (910) to spur growth and infection with any AA present in the production bioreactor.

Cell lines grown in mini bioreactors (910) for subsequent sampling with, for example, Radiance™ (920) can be used to test CM for AA, as shown in FIG. 9. Samples of CM are pumped into mini bioreactors from a large process bioreactor (900) that can then be sampled using LFC technology (920) (e.g., Radiance™) periodically to ascertain if adventitious agents are present. Multiple bioreactors can be used to sample at different time points in the production process if needed. The mini bioreactor(s) would, in aspects, have optical windows for spectroscopic analysis of cell lines for signs of infection that could be used to provide identification of virus infection or mycoplasma, or prions, or bacterial, fungal, or protozoan infection.

FIG. 10 shows the use of macrophage cells (white blood cells that engulf foreign material including viruses, bacteria, vegetative spores, and almost any other material), in this example as in vitro sentinel cells, for the detection of AA present in CM. The macrophages respond to the presence of foreign materials in unique ways detectable via LFC and can also engulf the foreign material (virus, viral inclusion bodies, bacterial spores or vegetative cells, exosomes, or any other biological material) thus increasing their refractive index by concentrating AA inside their membranes as they engulf them. This serves to increase the LFC response to AA and also to make the macrophages a convenient and detectable container or vehicle for LFC to sort and deliver preconcentrated AA to other techniques for further analysis. It will be important in this application to exclude the bioproduction cells (CHO or others) so they are not engulfed by the macrophages, influencing the assay outcome. Although presumably the CHO cells² would generally not be engulfed as they are the same size or larger than the macrophages³. Alternative macrophage activation (known activators such as plate binding, plate composition, media additives, addition of biomolecules including lipopolysaccharides (LPS), bacterial or viral proteins, among others) could be used to selectively control phagocytic activity or phenotypic state including changes in gene or protein expression.

Specificity in viral, bacterial, or other organism detection is made possible through the use of the many parameters that LFC/Radiance™ measures, including size, velocity (related to optical force), size normalized velocity, cellular volume, effective refractive index, eccentricity, deformability, cell granularity, rotation, orientation, optical complexity, membrane greyscale, or other parameters measured using LFC/Radiance™. This represents the use of multivariate parameter space including images to define classes of viruses or other organisms for AAT screening purposes. Coupling with optical spectroscopy would provide additional specificity including Raman, fluorescence, chemiluminescence, circular dichroism, or other methods.

The methods and devices described herein may be used in conjunction with the inventions described and claimed in U.S. patent application Ser. No. 15,853,763 (filed Dec. 23, 2017), Ser. No. 16/349,530 (filed Dec. 22, 2020), Ser. No. 16/982,935 (filed Sep. 21, 2020), Ser. No. 16/378,067 (filed Apr. 8, 2019), Ser. No. 17/016,079 (filed Sep. 9, 2020) and U.S. Provisional Patent Application Ser. No. 63/049,499 (filed Jul. 8, 2020), each of which is incorporated herein it its entirety.

One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

1. (canceled)

2. A microfluidic system comprising:
a reservoir comprising a solution;
a cell confinement region; and
a microfluidic device comprising a plurality of channels fluidically coupling the reservoir to the cell confinement region;
wherein the microfluidic system is configured to flow the solution through the cell confinement region.

3. The microfluidic system of claim 2, wherein the cell confinement region comprises a cell.

4. The microfluidic system of claim 3, wherein the microfluidic system is configured to prevent egress of the cell from the cell confinement region.

5. The microfluidic system of claim 3, wherein the microfluidic system is configured to prevent egress of the cell from the cell confinement region with an optical, magnetic, or electrokinetic force.

6. The microfluidic system of claim 3, wherein the cell confinement region is partitioned from the plurality of channels by a semipermeable barrier that prevents egress of the microcarrier, the cell growth disc, or the cell growth carrier from the cell confinement region.

7. The microfluidic system of claim 3, wherein the cell is coupled to a microcarrier, a cell growth disc, a cell growth carrier, or a surface of the cell confinement region.

8. The method of claim 7, wherein the microcarrier, the cell growth disc, the cell growth carrier, or the surface of the cell confinement region is dissolvable.

9. The microfluidic system of claim 7, wherein the microfluidic system is configured to prevent egress of the microcarrier, the cell growth disc, or the cell growth carrier from the cell confinement region.

10. The microfluidic system of claim 7, wherein the cell confinement region is partitioned from the plurality of channels by a semipermeable barrier that prevents egress of the microcarrier, the cell growth disc, or the cell growth carrier from the cell confinement region.

11. The microfluidic system of claim 10, wherein the semipermeable barrier comprises a membrane, a frit, a filter, a pillar, or a weir.

12. The microfluidic system of claim 3, wherein a diameter of an opening that fluidically couples the cell confinement region to the plurality of channels is smaller than a diameter of the cell.

13. The microfluidic system of claim 7, wherein a diameter of an opening that fluidically couples the cell confinement region to the plurality of channels is larger than a diameter of the microcarrier, the cell growth disc, or the cell growth carrier.

14. The microfluidic system of claim 2, wherein the microfluidic system comprises a pump or a syringe configured to flow the solution through the cell confinement region.

15. The microfluidic system of claim 2, wherein the microfluidic system comprises a plurality of cell confinement regions.

16. The microfluidic system of claim 15, wherein the plurality of cell confinement regions are connected in parallel, in series, or a combination thereof by the plurality of channels.

17. The microfluidic system of claim 2, wherein the microfluidic device further comprises a fluid reservoir that is fluidically coupled to the cell confinement region.

18. The microfluidic system of claim 17, wherein the fluid reservoir comprises a plurality of serpentine channels.

19. The microfluidic system of claim 17, wherein the microfluidic system is configured to prevent egress of the cells, the microcarrier, the cell growth disc, or the cell growth carrier from the cell confinement region into the fluid reservoir.

20. The microfluidic system of claim 2, wherein the cell confinement region is detachable from the microfluidic device.

21. The microfluidic system of claim 2, wherein the cell confinement region is configured to screw into the microfluidic device and fluidically couple to the plurality of channels.

22. The microfluidic system of claim 2, wherein the cell confinement region comprises an opening through which the cells, the microcarrier, the cell growth disc, or the cell growth carrier can be added.

23. The microfluidic system of claim 2, wherein the cell confinement region comprises a lower portion disposed within a flow path of the plurality of channels and an upper region that is not exposed to the flow path of the plurality of channels.

24. The microfluidic system of claim 23, wherein the flow path comprises a downward trajectory through the cell confinement region.

25. The microfluidic system of claim 2, wherein the solution is contained within oil within the reservoir.

26. The microfluidic system of claim 2, wherein the reservoir comprises a hydrophobic coating.

27. The microfluidic system of claim 7, wherein the surface of the cell confinement region comprises a porous support.

28. The microfluidic system of claim 2, wherein the solution comprises conditioned media.

29. The microfluidic system of claim 3, wherein the cell is configured to express a fluorescent protein upon contact to an analyte in the solution.

30. The microfluidic system of claim 29, wherein the analyte is a bacterium or a virus.

31. The microfluidic system of claim 2, further comprising an instrument configured to measure a cellular response.

32. The microfluidic system of claim 2, wherein the instrument is configured for single cell analysis, nucleic acid analysis, or protein analysis.

33. A microfluidic system comprising:
a reservoir comprising a solution;
a cell confinement region comprising cells coupled to a surface, a microcarrier, a cell growth disc, or a cell growth carrier;
a microfluidic device comprising a plurality of channels fluidically coupling the reservoir to the cell confinement region through at least one opening, wherein the at least one opening is configured to prevent egress of the cells, the microcarrier, the cell growth disc, or the cell growth carrier from the cell confinement region into the plurality of channels; and
a pump configured to flow the solution through the cell confinement region.

34. A method for measuring the concentration of an analyte in solution, comprising:
contacting cells with the solution, and
measuring a response of the cells to the solution.

35. The method of claim 34, wherein the contacting is performed within the cell confinement region of the microfluidic system of claim 2.

36. The method of claim 34, wherein the measuring comprises an optical force measurement, spectroscopy, mass spectrometry, single cell analysis, nucleic acid sequencing, or a combination thereof.

37. The method of claim 34, wherein the cells express a fluorescent protein upon contact to the solution, and wherein the measuring comprises detecting the fluorescent protein.

38. The method of claim 35, wherein the cell confinement region is detached from the microfluidic device prior to the measuring.

39. The method of claim 34, wherein the cells are added to the cell confinement region while frozen.
